(19) United States
(12) Patent Application Publication
(10) Pub. No.: US 2004/0228926 A1
(43) Pub. Date: Nov. 18, 2004

(54) Methods for treating cancer using perna canaliculus component(S) and extracts of perna canaliculus

(76) Inventors: Roger V. Kendall, Westford, VT (US); John Lawson, Clemson, SC (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/800,016

(22) Filed: Mar. 15, 2004

Related U.S. Application Data
(60) Provisional application No. 60/454,340, filed on Mar. 14, 2003.

(51) Int. Cl. A61K 35/56

(52) U.S. Cl. 424/547

(57) ABSTRACT

Described are methods for administering at least one component derived from Perna canaliculus or Mytilus edulis, particularly as an extract, to treat cancer and cancerous tumors in man or animals. Also described are novel compositions of extracts from Perna canaliculus or Mytilus edulis, methods of making these novel compositions, and the use of these compositions in the described methods.

This application claims the benefit of the filing date of U.S. Provisional Application Serial No. 60/454,340 filed Mar. 14, 2003.

The invention includes the use of at least one component derived from Perna canaliculus to treat cancer and/or cancerous tumors in man or animals. The invention also includes novel compositions of extracts from Perna canaliculus, methods of making these novel compositions, and the use of these compositions in the described methods. Components and extracts of Blue mussels, i.e., Mytilus edulis, can analogously be provided and used according to the invention and all references made herein to Perna canaliculus or PCE should be understood to include Mytilus edulis and components or extracts thereof.

### BACKGROUND OF INVENTION

The use of preparations made from Perna canaliculus for therapeutic effect extends back at least twenty-five years since the mid-seventies. The nutritional and therapeutic properties of freeze-dried Perna canaliculus in alleviating the symptoms of arthritis were reported in Croft, 1979, Relief From Arthritis (Thorsons Publishing Group, Rochester, Vt.). This article also remarks on prior investigations of potential anti-cancer effects from Perna canaliculus and that “no anti-cancer activity was noted.” Studies in both animal and human experiments have given mixed results, but indicate overall that certain components in the mussels were potentially helpful in relieving inflammation and pain associated with arthritis. See, for example: McFarlane, June 1975, New Zealand Medical Journal, pg. 569; Audeval and Bouchart, 1986, Gazette Medicale 93:111; Miller et al., 1993, Agents Actions 38:139; and, Whitehouse et al., 1997, Inflammopharmacology.

The analogy of the Mytilus edulis mussel to Perna canaliculus for anti-inflammatory purposes is discussed, for example, in Hoagland et al., Woods Hole Oceanographic Institute (WHOI Marine Policy Center, Oct. 21, 2001.

### SUMMARY OF THE INVENTION

It has been discovered that at least one component derived from Perna canaliculus (referred to herein as Perna canaliculus extract (PCE)) has activity in treating a wide range of cancer forms, particularly in exhibiting cytotoxicity against a wide range of cancer cells, i.e., malignant tumor cells.

Treatment, as used herein, pertains to the therapeutic administration of the compounds of the invention for the prevention, amelioration, or cure of disease.

The PCE component may be any therapeutically active component derived from the flesh of the mussel or its organs, which are suitable for use in the preparation of food supplements or pharmaceutical preparations. The invention contemplates the use of unrefined components of the mussel, such as whole mussel, or any therapeutically active extracts thereof. In particular, the PCE may be a preparation of whole mussel which has been freeze-dried and ground such as, for example, the Sea Mussel product available from FoodScience Corporation, Essex Junction, Vermont or the Perna product available from DaVinci Laboratories, Essex Junction, Vermont. Additionally, therapeutically active extracts of Perna canaliculus may also be employed. Such therapeutically active extracts have been described as follows: Macrides and Kalafatis, PCT/AU95/00485, WO 96/05164 (a purified lipid extract); Whitehouse et al., 1997, Inflammopharmacology 5:237-246 (a purified lipid extract); Kosuge and Sugiyama, U.S. Pat. No. 4,801,453 (stabilized mussel extract); Couch et al., 1982, The New Zealand Medical Journal 95:803 (a protein fraction); Miller et al., 1993, Agents Actions 38:139 (an aqueous fraction). The novel extracts described below are also useful in the methods.

As used herein, a therapeutically active PCE component according to this invention is a component that exhibits cytotoxicity to cancer cells, particularly malignant tumor cells, in humans or animals. For example, several cancer cell lines are known in the art for modeling anti-cancer efficacy and may be used to determine anti-cancer activities. Such models include, for example, potato tumor assay (which has been reported to have good agreement with the mouse 3PS anti-leukemia model; see, e.g., Galsky et al., J.Nat.Cancer Inst, 67, p. 689 (1981)), human osteosarcoma (MG-63), human cervical cancer (SiHa), human kidney tumor (BHK-21) and human monocytic leukemia cell lines. As part of the invention, it has been discovered that extracts containing Perna canaliculus components are active in inhibiting cell growth in each of these cell line models. Thus, the extracts containing a Perna canaliculus component model for activity, particularly in humans, against leukemia, osteosarcoma, cervical cancer, kidney tumors, and monocytic leukemia. Given this wide range of activity in differing cell lines, however, it can also be concluded that the Perna canaliculus component(s) have a broadly applicable anti-cancer activity, particularly anti-tumor. For example, the Perna canaliculus component(s) would be active against prostrate cancer and estrogen dependent or non-estrogen dependent breast cancers, melanoma, and bladder cancer, in addition to those indicated above.

The inhibition was shown, in each case, to occur only in the S-phase (growth phase) of the cell cycle, no inhibition occurring in the G₀ phase (resting phase). Though not intended to be bound by this theory, it is believed that, as with camptothecin shown in the Examples, the extract seems to target cell cycling at some unknown point and to destroy the cancer cells at that stage. Therefore, there is no lysis, i.e., from detergent or TWEEN activity. The addition of the extract to resting cells has no deleterious effects if diluted out prior to the cells entering the S-phase. In vivo, this indicates that there would be no damage to normal cells which are mostly in the resting phase.

The inhibitory activity was also shown to not be affected by prior treatment of the Perna canaliculus component(s) with proteolytic enzymes. See FIGS. 8 and 13. This is indicative that the factor(s) providing the inhibitory effect are not proteins. They could possibly be lipid or carbohydrate fractions, but this is not yet known.

Included as useful extracts with Perna canaliculus component(s) for the invention are novel extracts forming a further feature of this invention. These extracts are prepared by extracting ground freeze-dried whole Perna canaliculus mussel with a polyoxyethylene sorbitan ester non-ionic surfactant. Particularly preferred as the polyoxyethylene sorbitan ester are the polysorbates sold under the TWEEN designation, e.g., TWEEN 20, 40, 60, 65, 80 and 85. The extract is prepared by providing an aqueous solution of the ground freeze-dried whole Perna canaliculus mussel with the extracting agent, e.g., the TWEEN surfactant, agitating the mixture, centrifuging and then decanting one or more times to obtain the liquid portion, and then filtering one or more times through successively smaller filters (e.g., Millipore filter) to filter out small solids remaining in the liquid portion. The desired liquid extract can then, optionally, be filter-sterilized as a final step. The filter-sterilization involves passing through a filter of size sufficient to remove all forms of any living organism, including spores, for example a 0.22 um sterile filter, and passing into a sterile container for future use.

Another useful extract containing Perna canaliculus component(s) for the invention is a glycogen extract, such as supplied by Aroma NZ, Ltd. (Christchurch, New Zealand). This extract can be further processed by the above-described centrifuging and filtering steps.

In another aspect of the invention, an extract can be additionally purified of components in a certain molecular size range. This purification is preferably conducted by ultrafiltration methods. The ultrafiltration can be conducted upon any extract but ultrafiltration of the polyoxyethylene sorbitan ester non-ionic surfactant extract discussed above is preferred. The ultrafiltration is preferably conducted to remove smaller size materials. Thus, for example, the extract can be filtered by a 100 kD (kilodalton) filter, e.g., 100,000 MWCO, and the material blocked by the filter retained for use. In another embodiment, materials retained by a 300,000 MWCO filter or materials retained by a 100,000 MWCO but passing through a 300,000 MWCO are provided. The material retained is not necessarily only compounds with a molecular weight of 100 kD or more but may contain smaller molecular weight molecules which agglomerate into materials too large to pass the filter. Ultrafiltration apparatus known in the art can be used in a manner known in the art for the described filtering steps. For example, the ultrafiltration steps can include washing, backfiltering and elution steps.

In another aspect of the invention, the extracts are pH treated. The pH treatment can also be conducted in conjunction with the above-described purification by ultrafiltration treatments, either before or after the filtration. The pH treatment is conducted by mixing the extract (which is otherwise about neutral pH) with a source of H⁺ mions, for example HCl, to decrease the pH or OH⁻ ions, for example NaOH, to increase the pH. Such methods of altering pH are known in the art and variations may be employed. It is preferred if the pH is altered such that the extract has a neutral to basic pH, particularly pH 7-9. However, extracts altered to acidic pH also showed activity (see the following examples, particularly FIG. 6) demonstrating the efficacy of their use even when exposed to the acidic environment of the gastrointestinal tract.

As used herein, the term animal (to which the above-described methods can be applied) includes, but is not limited to, mammals. Preferred mammals for treatment according to the methods include humans, horses, farm animals and household pets.

The preferred route for administration of the Perna canaliculus component(s), particularly the described extracts, is via oral administration. For example, this does not require high sterility of the administration form. It can also be administered by intramuscular injection, intra peritoneal injection, parenteral administration, etc.

The Perna canaliculus component(s) used in this invention can be employed in admixture with conventional excipients, i.e. pharmaceutically acceptable organic or inorganic carrier substances suitable for parenteral, enteral (e.g., oral) application which do not deleteriously affect the active compound. Suitable pharmaceutically acceptable carriers include but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc. The pharmaceutical preparations can be sterilized and, if desired, mixed with auxiliary agents such as, for example, lubricants, preservatives, stabilizers, wetting agents, emulsifiers, salts for influencing osmotic pressure, buffers, coloring agents, flavoring agents and/or aromatic substances and the like which do not deleteriously affect the active compound. Other pharmaceutically acceptable carriers include aqueous solutions, non toxic excipients, including salts, preservatives, buffers and the like, as described for instance, in Remington 's Pharmaceutical Sciences, 18th ed. (1990), Mack Publishing Co., Easton, Pa., the contents of which are hereby incorporated by reference. The pH and exact concentration of the various components of the pharmaceutical composition are adjusted according to routine skills in the art. See Gilman et al. (eds.) (1990) Goodman and Gilman's: The Pharmacological Bases of Therapeutics, 8th ed., Pergamon Press.

The pharmaceutical compositions containing the Perna canaliculus component(s) are preferably prepared and administered in dose units. Solid dose units include tablets, capsules and suppositories. For treatment of a subject, depending on activity of the compound, manner of administration, nature and severity of the disorder, age and body weight of the patient, different daily doses may be necessary. Under certain circumstances, however, higher or lower daily doses may be appropriate. The average daily dosage of the compounds of this invention, when used for the described anti-cancer methods are not limited but, generally, the average daily dose is about 1 to about 500 mg/kg/day, preferably about 10 to about 100 mg/kg/day, more preferably about 20 to about 60 mg/kg/day (based on weight of ground whole mussel). When the Perna canaliculus component(s) are in extract form, the average daily dose will vary but can be readily determined by one of skill in the art. The daily dosage may be administered as a single dose per day or as a plurality of divided doses per day. The daily dosage may also be administered on a non-daily basis, such as for example, every second or third day, preferably such that an average daily dose as described above is achieved.

The Perna canaliculus component(s) can be administered concurrently or alternately with other therapeutic treatments conventionally employed for treating cancer. In a particular embodiment, the Perna canaliculus component(s), preferably as extracts, are co-administered with dimethylglycine (DMG). The use and activity of DMG is discussed, for example, in U.S. Pat. No. 4,994,492, which discussion is incorporated herein by reference.

Particular embodiments of the invention include the following:

a) a method of treating cancer comprising administering to the subject at least one Perna canaliculus or Mytilus edulis component.

a1) method a) where the cancer treated is leukemia, osteosarcoma, cervical cancer, kidney tumors, or monocytic leukemia in a human.

a2) method a) where the cancer treated is leukemia, osteosarcoma, cervical cancer, kidney tumors, monocytic leukemia, prostrate cancer, estrogen dependent or non-estrogen dependent breast cancer, melanoma, or bladder cancer, in a human.

b) methods a) where the Perna canaliculus or Mytilus edulis component is provided by freeze-dried ground whole mussel.

c) methods a) where the Perna canaliculus or Mytilus edulis component is an extract from freeze-dried ground whole mussel.

d) methods a) where the Perna canaliculus or Mytilus edulis component is an extract from freeze-dried ground whole mussel with a polyoxyethylene sorbitan ester non-ionic surfactant.

e) method d) where the extract is purified by ultrafiltration to remove materials of a size less than 100 kD.

f) method d) where the extract is purified by ultrafiltration to remove materials of a size less than 300 kD.

g) method d) where the extract is purified by ultrafiltration to remove materials of a size less than 100 kD and more than 300 kD.

f) method d) or e) where the extract is treated with an agent providing OH³¹ ions such that it has a basic pH.

g) method f) where the extract has a pH in the range from above 7 to 9.

h) method of one of a) to g) above wherein the component is a Perna canaliculus component.

i) a composition which comprises an extract of freeze-dried ground whole Perna canaliculus or Mytilus edulis mussel with a polyoxyethylene sorbitan ester non-ionic surfactant.

j) composition i) where the composition has been purified by ultrafiltration to remove materials of a size less than 100 kD.

k) composition i) where the composition has been purified by ultrafiltration to remove materials of a size less than 300 kD.

l) composition i) where the composition has been purified by ultrafiltration to remove materials of a size less than 100 kD and more than 300 kD.

m) composition i), j), k) or l) where the composition has a basic pH.

n) composition i), j), k) or l) where the composition has a pH in the range from above 7 to 9.

o) A method for preparing an extract containing components of Perna canaliculus or Mytilus edulis mussel which comprises providing an aqueous solution of the ground freeze-dried whole mussel with a polyoxyethylene sorbitan ester non-ionic surfactant, as extracting agent, agitating the mixture, centrifuging and then decanting one or more times to obtain the liquid portion and filtering one or more times to remove small solids remaining in the liquid portion.

p) method o) further comprising filter sterilizing the extract by passing it through a filter of a size sufficient to remove all forms of any living organism, including spores.

q) method o) or p) further comprising subjecting the extract to ultrafiltration to remove components of a particular size range.

r) method o) or p) further comprising subjecting the extract to ultrafiltration to remove components of a size less than 100 kD.

s) method o) or p) further comprising subjecting the extract to ultrafiltration to remove components of a size less than 300 kD.

t) method o) or p) further comprising subjecting the extract to ultrafiltration to remove components of a size less than 100 kD and more than 300 kD.

u) method o), p), q), r), s) or t) further comprising treating the extract with an agent providing OH⁻ ions to increase its pH.

v) method u) wherein the pH is increased to a basic pH.

w) method u) wherein the pH is increased to from above 7 to 9.

The entire disclosure of all applications, patents and publications, cited above and below and of U.S. Provisional Application No. 60/454,340, filed Mar. 14, 2003, are hereby incorporated by reference.

### BRIEF DESCRIPTION OF THE DRAWINGS

FIG. 1. 50% inhibition of Cox-1 is seen between the 1:200 and 1:400 dilutions of the Tween extract and between the 1:10 and 1:100 dilutions of the Glycogen extract.

FIG. 2. 50% inhibition of Cox-2 is seen between the 1:200 and 1:400 dilutions of the Tween extract and between the 1:10 and 1:100 dilutions of the Glycogen extract.

FIG. 3. Significant inhibition of tumors is seen at the 1:10 and 1:00 dilutions of Tween extract.

FIG. 4. Significant inhibition of potato tumors is seen with the 1:10 concentration of the Glycogen extract.

FIG. 5. The fraction of Tween extract retained by the 100K filter shows significant inhibition of potato tumors at both the 1:10 and 1:100 concentrations.

FIG. 6. The pH of the Tween extract is altered using 10N NaOH and 10N HCl. Significant inhibition of potato tumors occurs at both the 1:10 and 1:100 concentrations of the pH 2>100 K sample, at the 1:10 concentration of the pH 2 100K-10K sample, at the 1:10 and 1:100 concentrations of the pH 2<10K sample, and at the 1:10 and 1:100 concentrations of the pH 9>100 K sample.

FIG. 7. The pH of the Tween extract is altered using 1 N NaOH before filtering. These extracts are tested at a concentration of 1:10. Significant inhibition of potato tumors is seen at the pH 9>100 K sample, the pH 8>100 K sample, and the pH 7>100K sample.

FIG. 8. The Tween extract is treated with Pronase and Proteinase K independently and incubated at 37 degrees C for time periods ranging from 0-48 hours. The enzyme activity is halted by incubating tubes at 80 degrees C for 15 minutes. Tx is untreated full strength Tween extract that is incubated along with the other samples for 48 hours. Samples are tested at a concentration of 1:10. No significant change in activity is seen in any sample upon treatment with either proteolytic enzyme.

FIG. 9. Significant inhibition of tumors is seen with the >300K and 300K-100K fractions of the Tween extract. Significant inhibition of tumors is seen with the >300K fraction of the Glycogen extract. Campto is 0.1 ppm Camptothecin.

FIG. 10. The fraction of glycogen extract retained by the 100K filter shows significant inhibition of potato tumors at both the 1:10 and 1:100 concentrations. The fraction of glycogen extract that passed through the 100K filter but was retained by the 30K filter shows slightly significant inhibition of potato tumors at both the 1:10 and 1:100 concentrations.

FIG. 11. The pH of the glycogen extract is altered using 10 N NaOH and 10 N HCl before filtering. Significant inhibition of potato tumors is seen at the 1:10 and 1:100 concentrations of the pH 2>100 K sample, at the 1:10 and 1:100 concentrations of the pH 9>100 K sample, and at the 1:10 concentration of the pH 9<10K sample

FIG. 12. The pH of the glycogen extract is altered using 1 N NaOH before filtering. These extracts are tested at a concentration of 1:10. Significant inhibition of potato tumors is seen at the pH 9>100 K sample, the pH 9 100K-10K sample, the pH 8>100 K sample, the pH 7>100K sample, and the pH 7 100K-10K sample.

FIG. 13. The glycogen extract is treated with Pronase and Proteinase K independently and incubated at 37 degrees C for time periods ranging from 0-48 hours. The enzyme activity is halted by incubating tubes at 80 degrees C for 15 minutes. Gx is untreated full strength glycogen extract that was incubated along with the other samples for 48 hours. Samples were tested at a concentration of 1:10. No significant change in activity is seen in any sample upon treatment with either proteolytic enzyme.

FIG. 14. Pena extracts at the indicated % concentrations are shown to inhibit cervical carcinoma (SiHa) cells.

FIG. 15. Perna extracts at the indicated % concentrations are shown to inhibit osteocarcinoma cells (MG-63).

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure.

### EXAMPLES

In the following examples, all parts and percentages are by weight unless otherwise indicated.

### Example 1

### Preparation of a Therapeutically Active PCE Component

A therapeutically active formulation of Perna canaliculus is prepared by freeze-drying the flesh of the mussel and grinding it into a powder. The product is formulated into capsules with the excipients of alfalfa, cellulose and magnesium stearate.

### Example 2

### Preparation of a TWEEN Extract of Perna canaliculus

Twenty grams of crude Perna powder (FoodScience Corp.) are placed in a 500 ml flask with 100 ml of 0.1% polysorbate-20 (TWEEN-20) solution in distilled water. The mixture is agitated for 24 hours at four degrees Celsius to provide a Perna slurry. The slurry is centrifuged for 20 minutes at 4000×g. The liquid portion is decanted and re-centrifuged in one hour intervals until no further pellet was formed. The liquid sample is then filtered using a 47 mm Millipore setup through a 1.2 mm filter, then a 0.8 mm filter, then a 0.45 mm filter and finally through a 0.22 mm filter. The liquid sample is then filtered sterilized using a 0.22 filter and sterile storage container. The extract is then tested for protein content using the Bio-Rad DC protein assay. Dilutions of 1:10, 1:100, 1:200, 1:400, 1:500, 1:800, 1:1000 and 1:1600 in distilled water are provided.

### Example 3

### Preparation of a Glycogen Extract of Perna canaliculus

An experimental glycogen extract from Perna is obtained from FoodScience Corp. One gram of the glycogen powder is mixed with 10 ml of distilled water and processed in the same manner as the Perna slurry in Example 2. Dilutions of 1:10, 1:100, 1:200, 1:400, 1:500, 1:800, 1:1000 and 1:1600 in distilled water were provided.

### Example 4

### Preparation of a TWEEN Extract of Perna canaliculus with Filtration

Variations of the TWEEN extract are created by using centrifugal filters with molecular weight cutoffs of 100 kD, 300 kD, and 10 kD. Fractions of less than 10 kD, between 10-100 kD, more than 100 kD, between 100-300 kD and more than 300 kD are obtained and tested, as described below.

### Example 5

### Preparation of a TWEEN Extract of Perna canaliculus with Filtration and pH Treatment

Variations of the TWEEN extract are obtained by pH alteration prior to filtration conducted in the above-discussed manner. The pH is raised by addition of NaOH or lowered by the addition of HCl. Extracts at pH 2, 7, 8 and 9 are prepared and tested.

### Example 6

### Cyclooxygenase (COX) Enzyme Inhibition

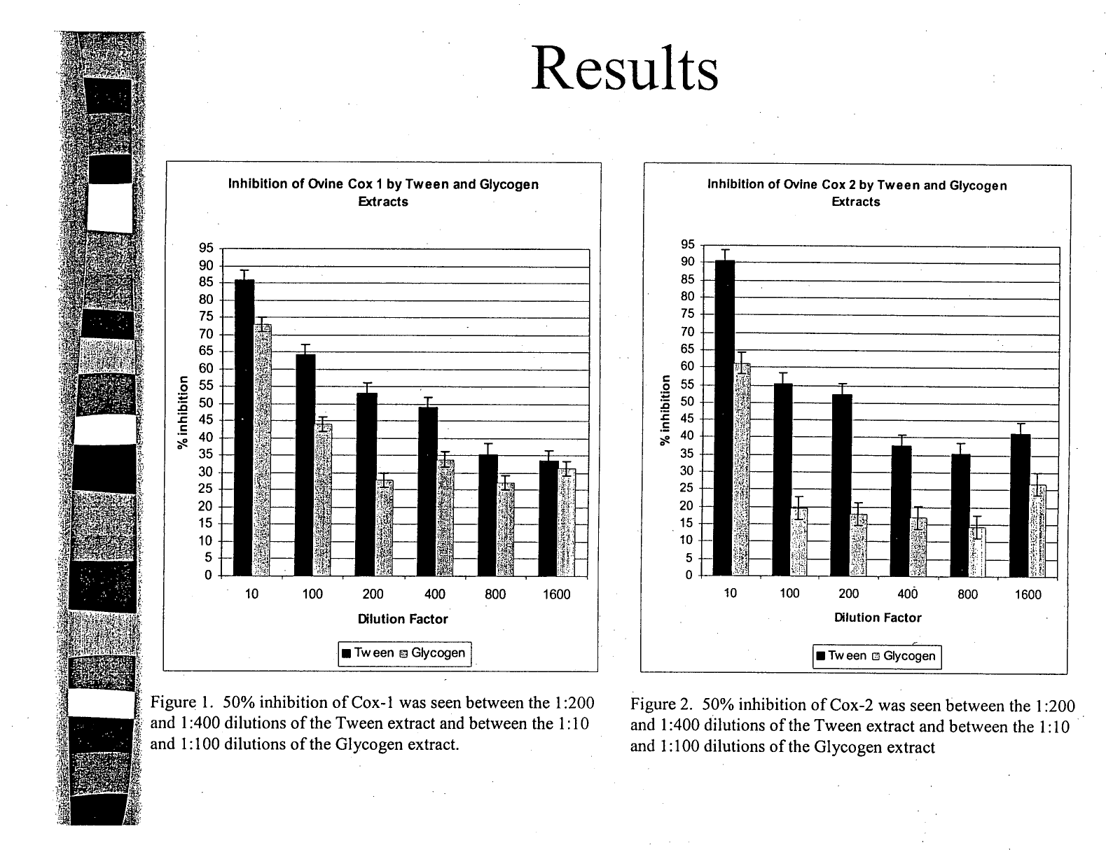

Perna extracts are tested in a colorimetric Cox (ovine) inhibitor-screening assay. Extracts are lyophilized and resuspended at the desired concentrations in DMSO. Analysis is done using the Colorimetric COX (ovine) Inhibitor Screening Assay (760111) from Cayman Chemical Company. Results are read using a micro plate reader at 595 nm. Initial results showed significant inhibition of both the Cox-1 and Cox-2 enzymes in vitro. See FIGS. 1 and 2.

### Example 7

### Potato Tumor Assays

Agrobacteria tumefaciens (Strain B6) is cultured on YEM plates for 48 hours at 28 degrees Celsius. Russet potatoes are cleaned and disinfested with Clorox. Cylinders are cut from the potatoes with a sterile cork bore (10 mm) and placed in sterile pH 4 distilled water. Disks approximately 0.5 cm thick are cut from the cylinders using a sterile scalpel and were placed in 24-well culture plates containing approximately 1 ml of 15% water agar in each well.

A standardized solution of A. tumefaciens is made in phosphate buffered saline (pH 7.2) to 1×10⁹ CFU/ml. Extracts and a Camptothecin control are prepared by adding (1) 400 micro liters of either extract or 0.1 ppm Camptothecin, (2) 100 micro liters water, and (3) 500 micro liters A. tumefaciens suspension. Each potato disk in the 24-well culture plate is then overlaid with 50 micro liters of the appropriate extract/water/bacteria mix and incubated at room temperature for 12 days.

On day 12 the potato disks are stained with Lugol's reagent and the tumors are counted.

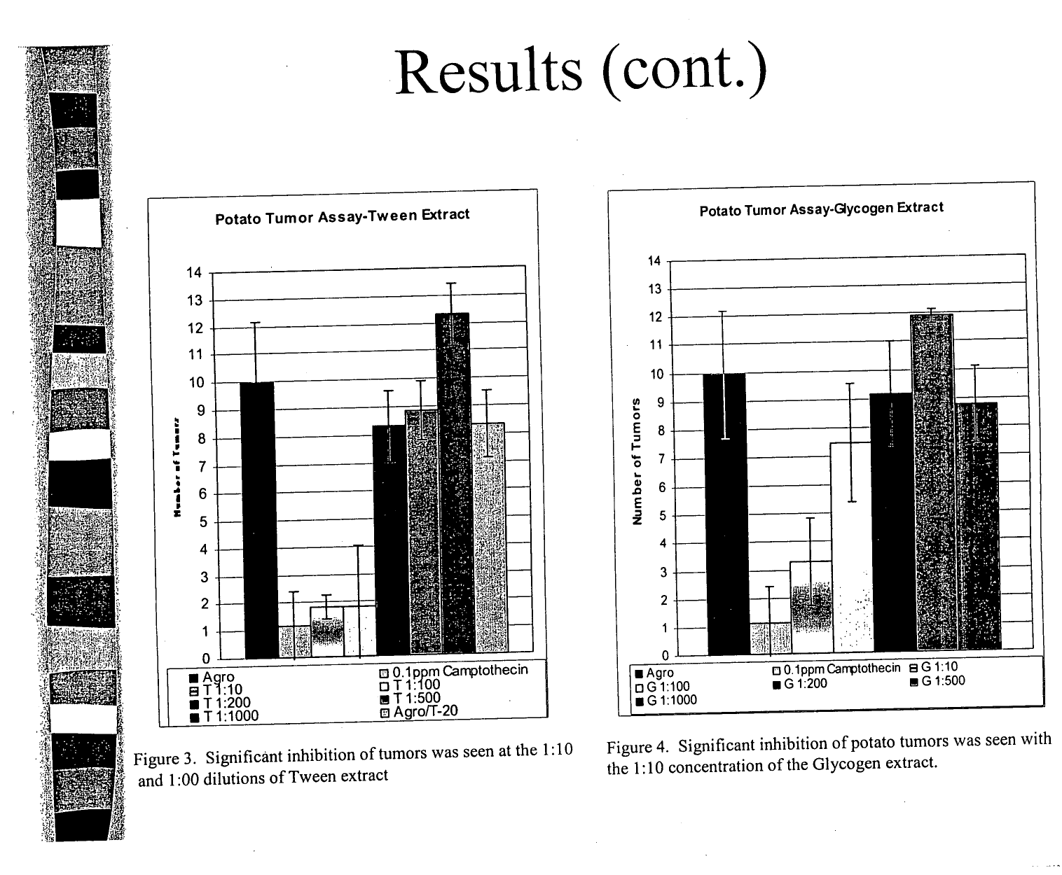

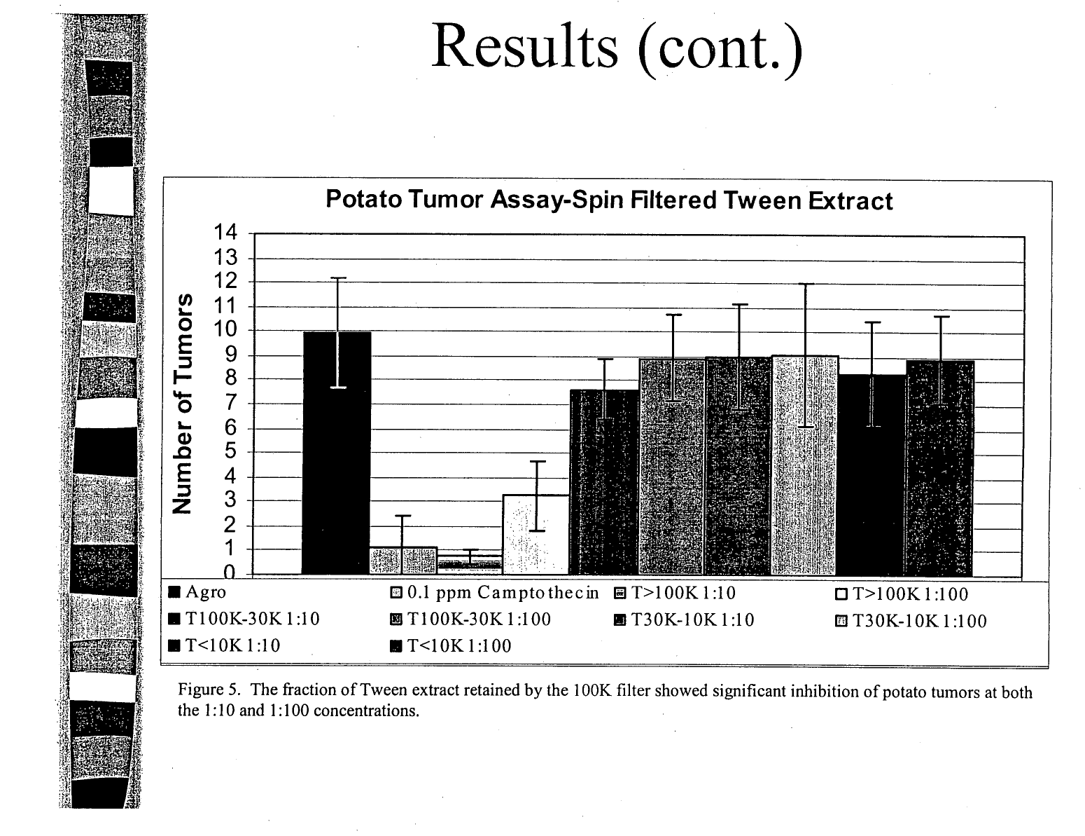

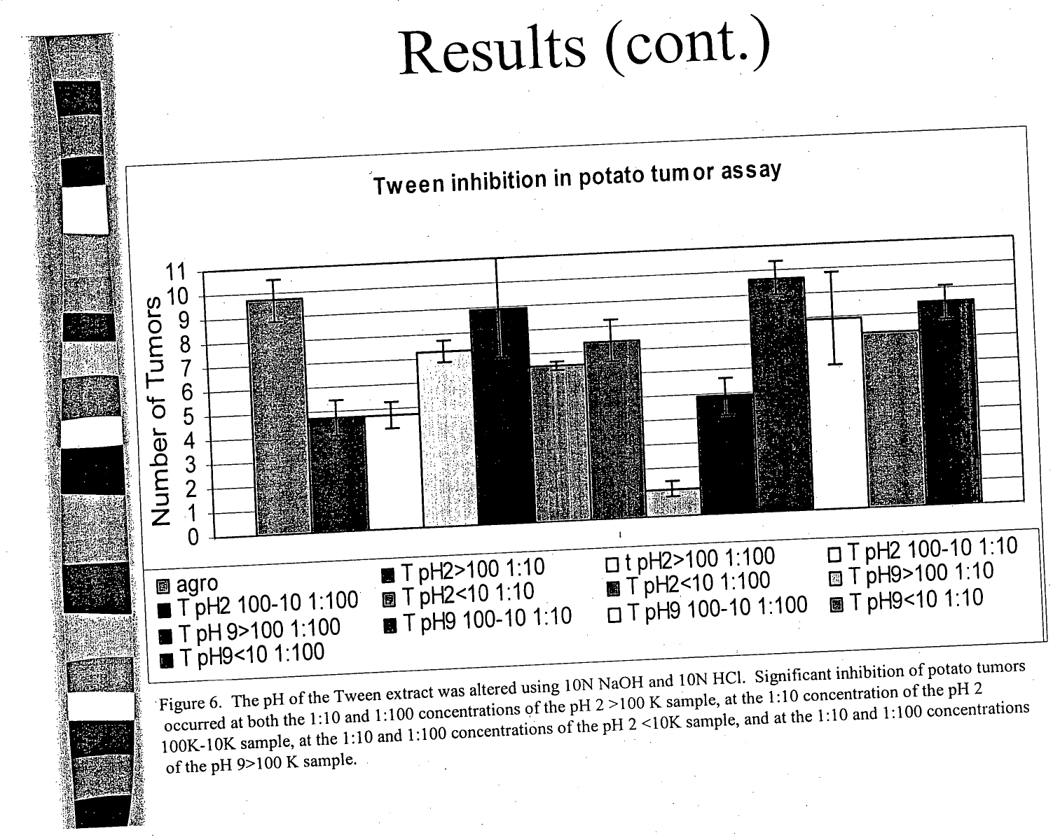

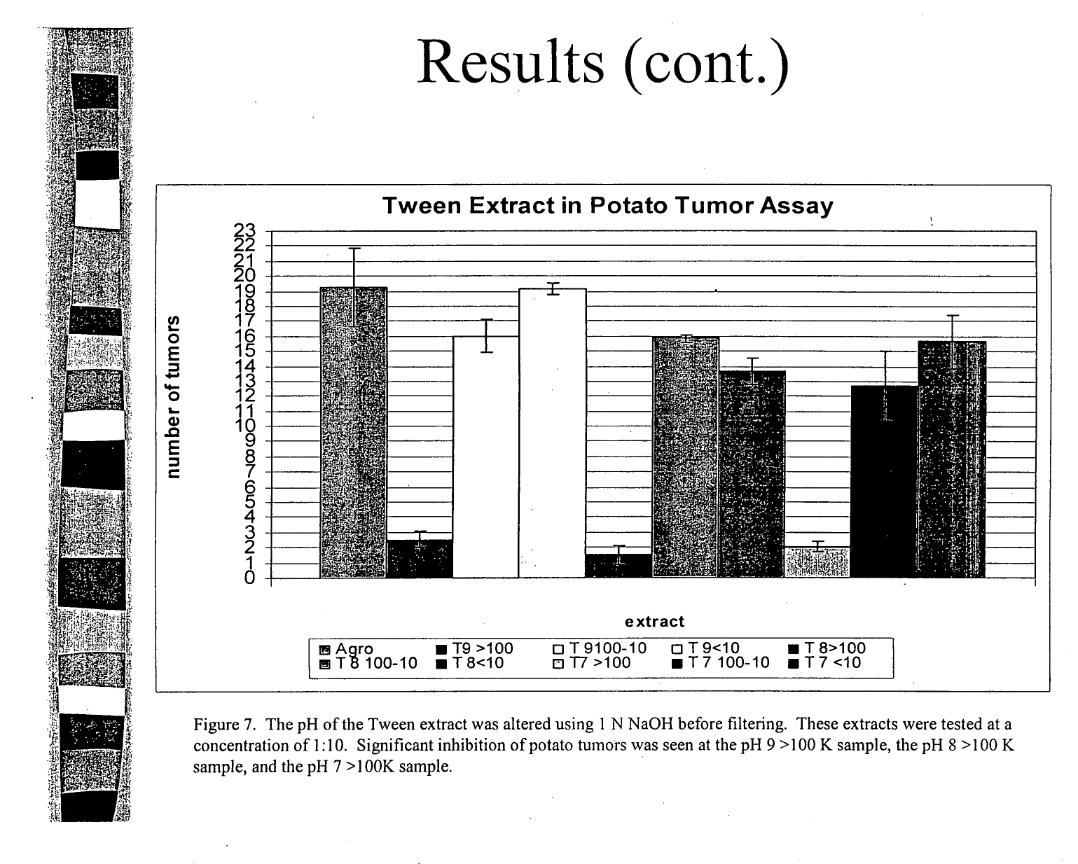

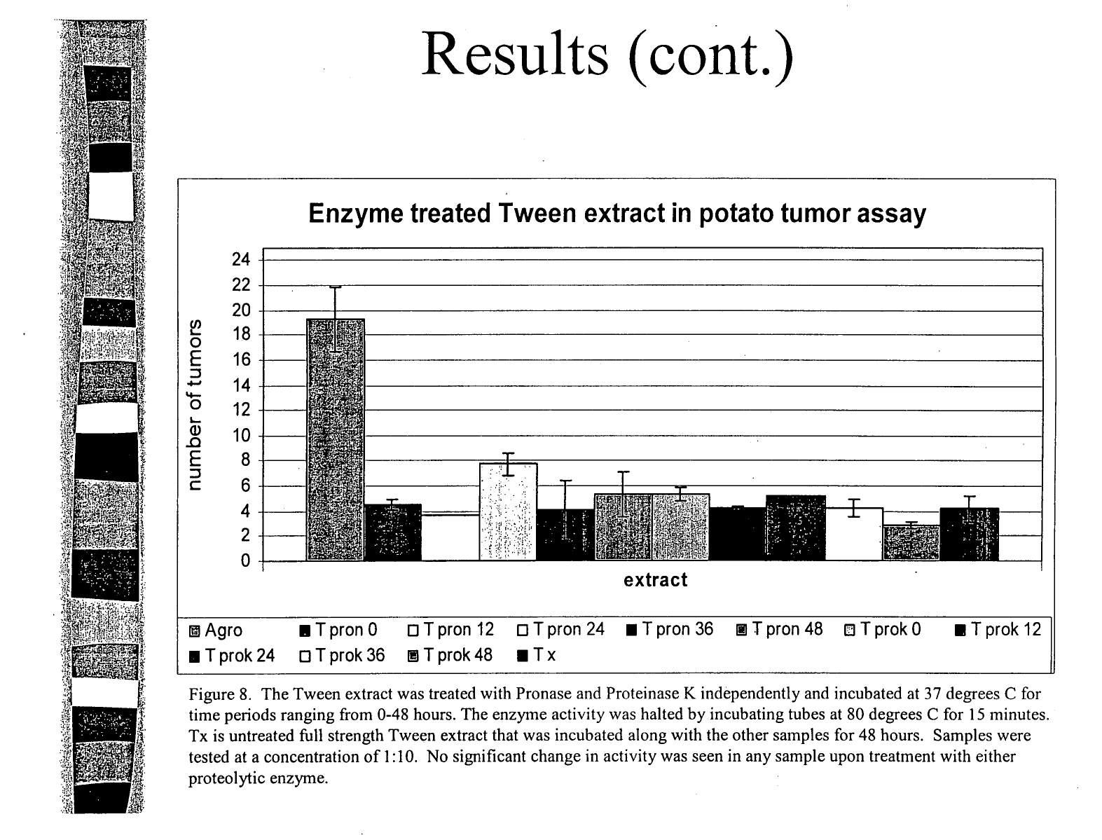

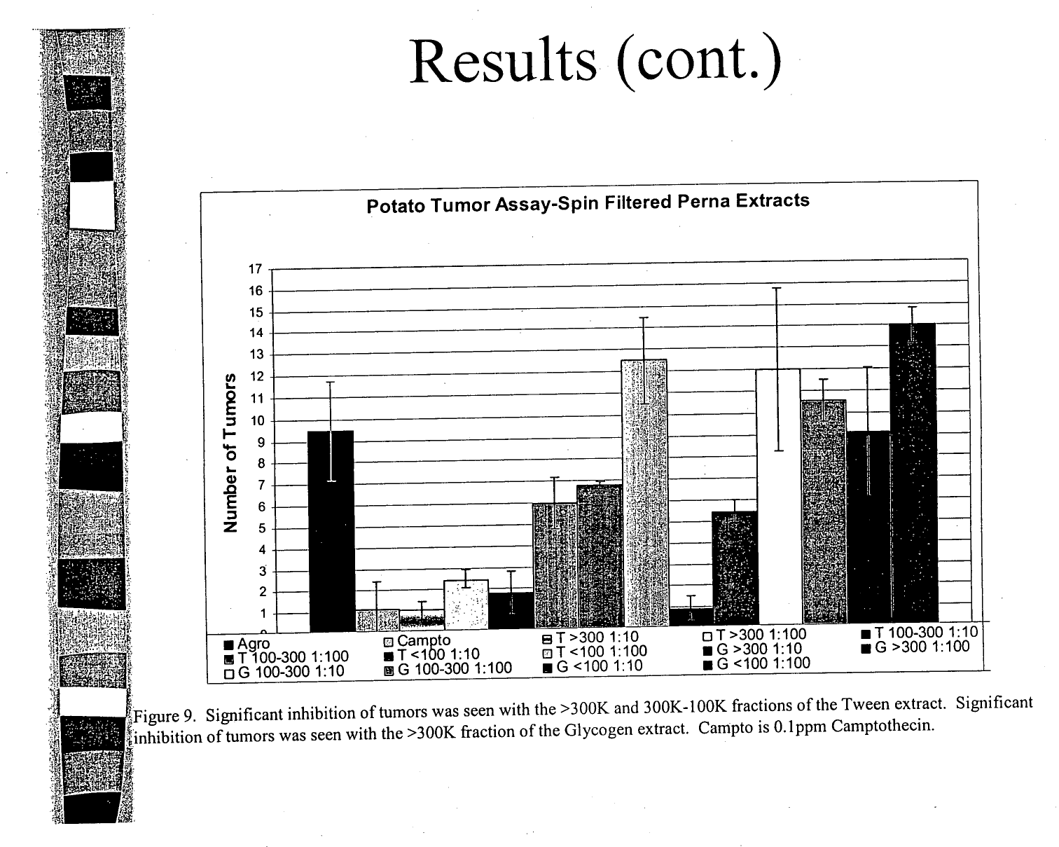

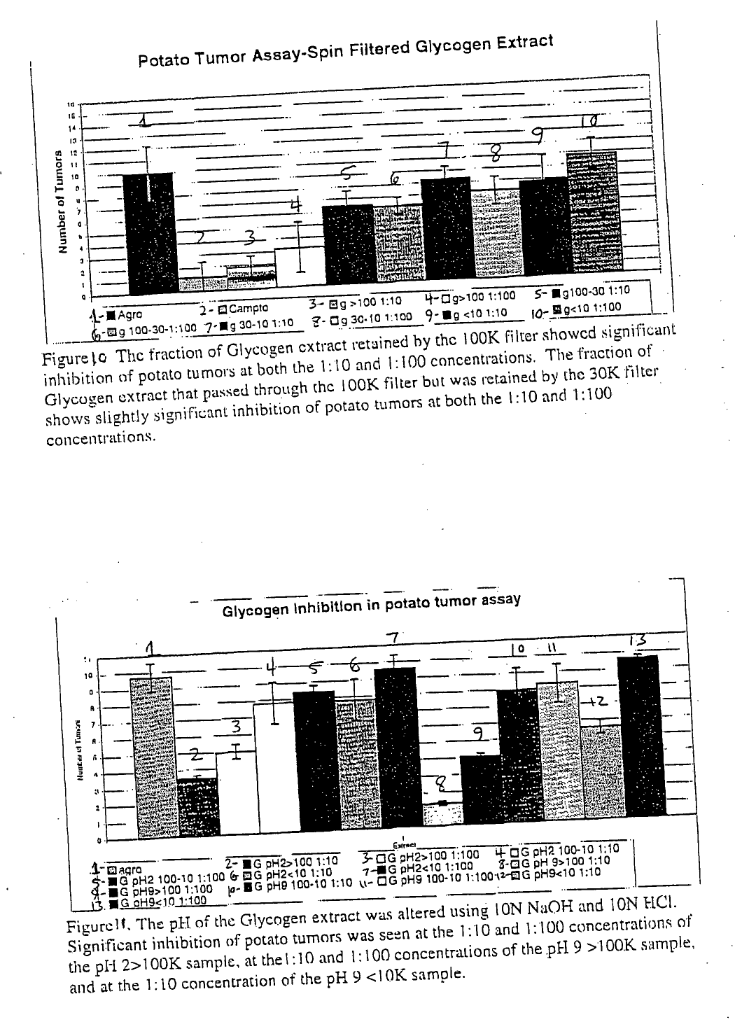

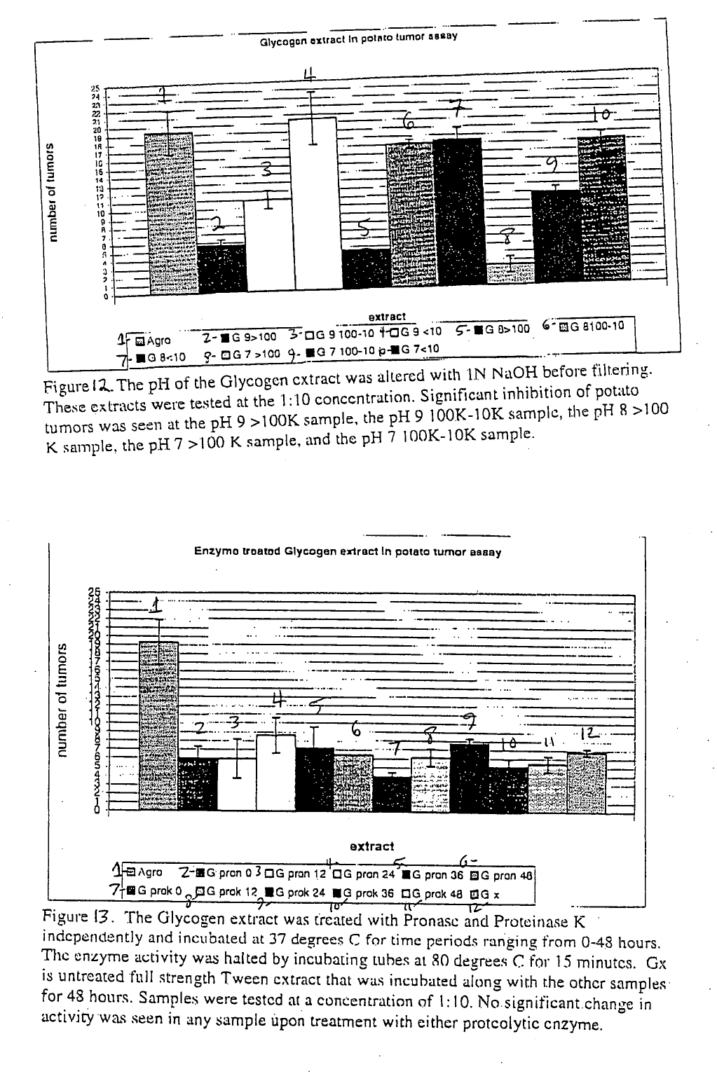

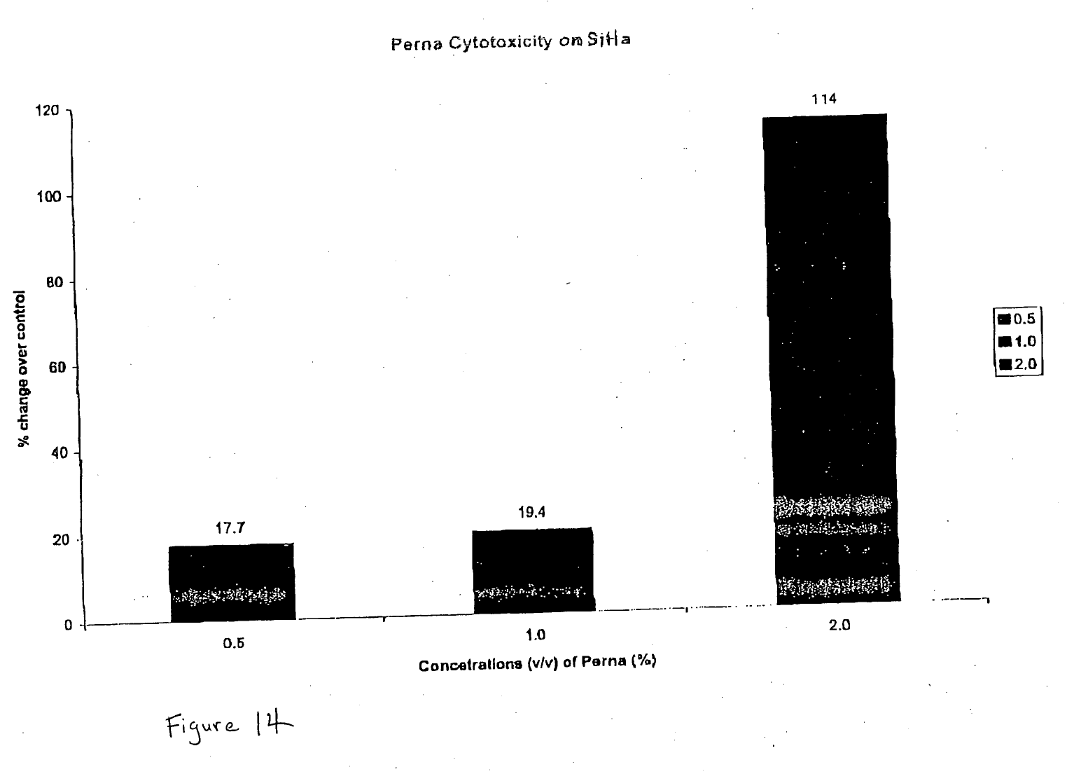

The activity of TWEEN and glycogen extracts in the potato tumor assay at various concentrations, purifications, pHs and enzyme-treated is shown in FIGS. 3-13.

### Example 8

### Perna Cytotoxicity on MG-63 Osteocarcinoma Cell Line Model

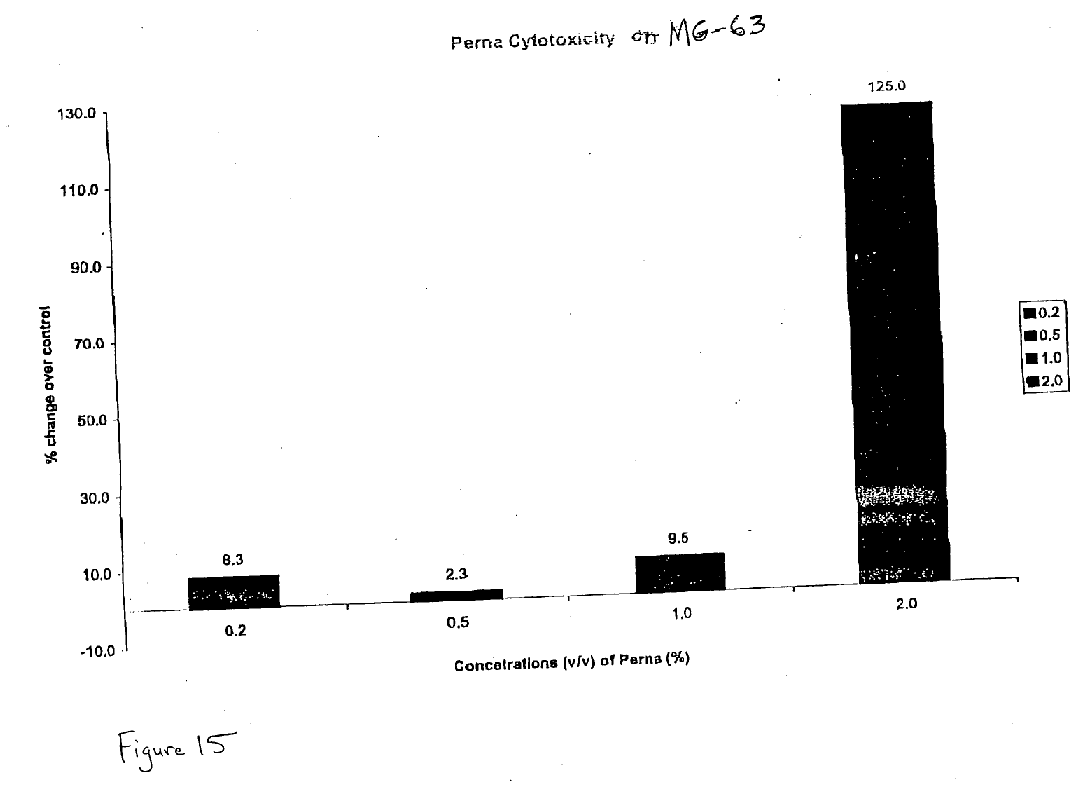

Tween extracts of Perna canaliculus at the concentrations indicated in FIG. 14 are shown to inhibit cervical carcinoma (SiHa) cells to the extent shown in the Figure. Cell death is demonstrated by calorimetric metabolic tests which detect changes in cellular respiratory activity.

### Example 9

### Perna Cytotoxicity on SiHa Cervical Carcinoma Cell Line Model

Tween extracts of Perna canaliculus at the concentrations indicated in FIG. 15 are shown to inhibit osteocarcinoma (MG-63) cells to the extent shown in the Figure. Cell death is demonstrated by calorimetric metabolic tests which detect changes in cellular respiratory activity.

### Example 10

### Perna Cytotoxicity on BHK-21 Kidney Cancer Cell Line Model

At concentrations varying from 2% down to vanishing dilutions of up to 1000%, the TWEEN Perna canaliculus extracts completely inhibit all growth of the BHK-21 cells, as demonstrated by calorimetric metabolic tests. Thus, no figure is necessary to demonstrate this.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

We claim:

1. A method of treating a malignant tumor cancer comprising administering to a subject at least one Perna canaliculus or Mytilus edulis mussel component which exhibits cytotoxicity to malignant tumor cells.

2. The method of claim 1, wherein the cancer treated is leukemia, osteosarcoma, cervical cancer, kidney tumors, monocytic leukemia, prostrate cancer, estrogen dependent or non-estrogen dependent breast cancer, melanoma, or bladder cancer, in a human.

3. The method of claim 1, wherein the cancer treated is leukemia, osteosarcoma, cervical cancer, kidney tumors, or monocytic leukemia in a human.

4. The method of claim 1, wherein the at least one Perna canaliculus or Mytilus edulis mussel component is provided as freeze-dried ground whole mussel.

5. The method of claim 1, wherein the at least one Perna canaliculus or Mytilus edulis mussel component is provided as an extract from freeze-dried ground whole mussel.

6. The method of claim 1, wherein the at least one Perna canaliculus or Mytilus edulis mussel component is provided as an extract from freeze-dried ground whole mussel extracted with a polyoxyethylene sorbitan ester non-ionic surfactant.

7. The method of claim 6, wherein the extract is purified by ultrafiltration to remove materials of a size less than 100 kD.

8. The method of claim 6, wherein the extract is purified by ultrafiltration to remove materials of a size less than 300 kD.

9. The method of claim 6, wherein the extract is purified by ultrafiltration to remove materials of a size less than 100 kD and materials of a size more than 300 kD.

10. The method of claim 6, wherein the extract is treated with an agent providing OH⁻ ions such that it has a basic pH.

11. The method of claim 6, wherein the extract has a pH in the range from above 7 to 9.

12. The method of claim 1, wherein the component is a Perna canaliculus mussel component.

13. A composition which comprises an extract of freeze-dried ground whole Perna canaliculus or Mytilus edulis mussel which has been extracted with a polyoxyethylene sorbitan ester non-ionic surfactant.

14. The composition of claim 13, wherein the composition has been purified by ultrafiltration to remove materials of a size less than 100 kD.

15. The composition of claim 13, wherein the composition has been purified by ultrafiltration to remove materials of a size less than 300 kD.

16. The composition of claim 13, wherein the composition has been purified by ultrafiltration to remove materials of a size less than 100 kD and materials of a size more than 300 kD.

17. The composition of claim 13, wherein the composition has a basic pH.

18. The composition of claim 13, wherein the composition has a pH in the range from above 7 to 9.

19. A method for preparing an extract containing at least one component of Perna canaliculus or Mytilus edulis mussel which comprises:
providing an aqueous solution of the ground freeze-dried whole mussel with a polyoxyethylene sorbitan ester non-ionic surfactant, as extracting agent,
agitating the mixture,
centrifuging the mixture,
then decanting one or more times to obtain the liquid portion, as the extract, and
optionally, filtering one or more times to remove small solids remaining in the liquid portion extract.

20. The method of claim 19, further comprising filter sterilizing the extract by passing it through a filter of a size sufficient to remove all forms of any living organism, including spores.

21. The method of claim 19, further comprising subjecting the extract to ultrafiltration to remove components of a particular size range.

22. The method of claim 19, further comprising subjecting the extract to ultrafiltration to remove components of a size less than 100 kD.

23. The method of claim 19, further comprising subjecting the extract to ultrafiltration to remove components of a size less than 300 kD.

24. The method of claim 19, further comprising subjecting the extract to ultrafiltration to remove components of a size less than 100 kD and components of a size more than 300 kD.

25. The method of claim 19, further comprising treating the extract with an agent providing OH⁻ions to increase its pH.

26. The method of claim 25, wherein the pH is increased to a pH in the range from above 7 to 9.
